(19) United States
(12) Patent Application Publication
(10) Pub. No.: US 2005/0048016 A1
(43) Pub. Date: Mar. 3, 2005

(54) Composition containing a silicone elastomer and a block silicone copolymer

(75) Inventors: Francoise Lebreton, Bures-Sur-Yvette (FR); Patricia Pierre, Antony (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/876,685

(22) Filed: Jun. 28, 2004

Related U.S. Application Data
(60) Provisional application No. 60/494,563, filed on Aug. 13, 2003.

(30) Foreign Application Priority Data
Jul. 2, 2003 (FR) 0308028

(51) Int. Cl. A61K 7/06; A61K 7/11

(52) U.S. Cl. 424/70.12

(57) ABSTRACT

The present patent application relates to a composition containing at least one elastomeric solid organopolysiloxane, at least one filler, at least one C₂-C₃ primary alcohol and an aqueous dispersion of at least one substantially linear block silicone copolymer.

### REFERENCE TO PRIOR APPLICATIONS

This application claims priority to U.S. provisional application 60/494,563 filed Apr. Aug. 13, 2003, and to French patent application 0308028 filed Jul. 2, 2003, both incorporated herein by reference.

### FIELD OF THE INVENTION

The present patent application relates to a composition, preferably one suitable for topical application, comprising an elastomeric solid organopolysiloxane, a filler, a primary alcohol and an aqueous dispersion of a block silicone copolymer, and to the uses of the composition, especially in cosmetics, in particular for making the skin matt and for fading out skin relief defects such as microreliefs, wrinkles and pores while at the same time making the skin look natural. The invention composition is preferably an oil-in-water emulsion. In addition, the compositions of the invention give a good “soft-focus” effect while at the same time being emollient and fresh.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Products known as “skin perfectors” are increasingly being sought by consumers. They are skincare or makeup compositions with matting properties, generally used to solve the sheen problems caused by excess sebum and to improve the long-term staying power of makeup, which has a tendency to become visually degraded in the course of the day. These compositions are particularly sought for users with combination skin or greasy skin, and also users in hot and humid climates. These matting compositions give the skin a matt appearance resulting from light-scattering power at the surface of the skin; this is known as the “soft focus” effect. A composition with “soft-focus” properties is such that, when it is applied to the skin, it gives a hazy effect which camouflages the microreliefs and other skin imperfections.

Document EP-A-790 055 discloses matting compositions containing a combination of an elastomeric solid organopolysiloxane with a fatty phase, giving the skin a long-lasting matt appearance.

To provide a good matting effect, the organopolysiloxane is often used in large amounts, for example at least 2% by weight of active material. In addition, it is often combined with fillers to improve the optical effects and/or sensory properties of the composition. However, the composition obtained then gives products that, when applied to the skin, have an excessively dry effect and give excessively dry deposits. The addition of emollient oils and high molecular weight moisturizers affords increased moisturizing and emollient properties. Nevertheless, this addition is accompanied by heaviness and thickness of the texture, and thus causes a loss of the desired primary qualities, namely lightness and an evanescent effect.

### SUMMARY OF THE INVENTION

There is thus still a need for a matting composition that has a good “soft-focus” effect while at the same time being pleasant to use, i.e. light, and not having a dry or greasy effect when applied to the skin.

The inventors have found, surprisingly, that the incorporation of particles of a substantially linear block silicone copolymer in aqueous dispersion and of a primary alcohol into compositions containing an elastomeric organopolysiloxane and fillers affords surprising moisturizing and emollient properties, combined with light, velvety and evanescent textures.

The composition thus obtained has the advantage of being both light and film-forming, of spreading perfectly on the skin without having a tacky or greasy effect, and of affording an emollient effect. The skin onto which the composition has been applied is supple, elastic and matt, the optical effect of the composition also allowing skin imperfections to be masked.

### DETAILED DESCRIPTION OF THE PREFERRED EMBODIMENTS

Thus, one subject of the present invention is a composition preferably suitable for topical application, comprising at least one elastomeric solid organopolysiloxane, at least one filler, at least one C₂-C₃ primary alcohol and an aqueous dispersion of at least one substantially linear block silicone copolymer.

Since the composition of the invention is intended especially for topical application, it preferably comprises a physiologically acceptable medium, i.e. a medium that is compatible with any keratin material such as the skin, the nails, mucous membranes and the hair or any other area of body skin. It may especially constitute a cosmetic or dermatological composition.

Substantially Linear Block Silicone Copolymer

The silicone copolymer constituting the globules or particles dispersed in the aqueous phase is a substantially linear block copolymer, i.e. a non-crosslinked copolymer, obtained by chain extension and not by crosslinking.

The aqueous dispersion of block copolymer particles is a silicone-in-water (Sil/W) emulsion, the oily globules of which consist of a high-viscosity silicone, such that these globules appear to form like “soft particles”.

The size of the substantially linear block silicone copolymer particles may vary widely. In a preferred manner in the present patent application, the substantially linear block silicone copolymer particles generally have a number-average size of less than or equal to 2 microns and preferably less than or equal to 1 micron.

The aqueous dispersions of substantially linear block silicone copolymer particles used in the composition according to the invention may be chosen especially from those described in document EP-A-874 017, the teaching of which is incorporated herein by reference. According to the document, the silicone copolymers constituting these particles may especially be obtained by a chain-extension reaction, in the presence of a catalyst, starting with at least:
(a) one polysiloxane (i) having at least one reactive group and preferably one or two reactive groups per molecule; and
(b) one organosilicon compound (ii) that reacts with the polysiloxane (i) via a chain-extension reaction.

In particular, the polysiloxane (i) is chosen from the compounds of formula (I): 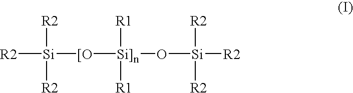
in which R₁ and R₂, independently of each other, represent a hydrocarbon-based group containing from 1 to 20 carbon atoms and preferably from 1 to 10 carbon atoms, such as methyl, ethyl, propyl or butyl, or an aryl group such as phenyl, or a reactive group, n is an integer greater than 1, with the proviso that there are on average between one and two reactive groups per polymer.

The term “reactive group” means any group capable of reacting with the organosilicon compound (ii) to form a block copolymer. Reactive groups that may be mentioned include hydrogen; aliphatically unsaturated groups and especially vinyl, allyl or hexanyl; a hydroxyl group; alkyoxy groups such as methoxy, ethoxy or propoxy; alkoxyalkoxy groups; an acetoxy group; amino groups, and mixtures thereof. Preferably, more than 90% and better still more than 98% of the reactive groups are at the end of a chain, i.e. the radicals R₂ generally constitute more than 90% and even 98% of the reactive groups.

Preferably, n is such that the polysiloxanes have a viscosity ranging from about 1 to 1×10⁶ mm²/sec at 25° C. n may especially be an integer ranging from 5 to 30, preferably from 10 to 30 and better still from 15 to 25.

The polysiloxanes of formula (I) are substantially linear polymers, i.e. polymers comprising few branches, and generally less than 2 mol % of siloxane units. Moreover, the groups R₁ and R₂ may optionally be substituted with amino groups, epoxy groups or groups comprising sulphur, silicon or oxygen.

Preferably, at least 80% of the groups R₁ are alkyl groups and better still methyl groups.

Preferably, the reactive group R₂ at a chain end is an aliphatically unsaturated group and especially vinyl.

Polysiloxanes (i) that may especially be mentioned include dimethylvinylsiloxypolydimethylsiloxane, a compound of formula (I) in which the radicals R₁ are methyl radicals and the radicals R₂ at the chain ends are vinyl radicals, whereas the other two radicals R₂ are methyl radicals.

The organosilicon compound (ii) may be chosen from the polysiloxanes of formula (I) or compounds acting as chain extenders. If it is a compound of formula (I), the polysiloxane (i) will comprise a first reactive group and the organosilicon compound (ii) will comprise a second reactive group that will react with the first. If it is a chain extender, this may be a silane, a siloxane (disiloxane or trisiloxane) or a silazane. The organosilicon compound (ii) is preferably a liquid organohydrogenopolysiloxane of formula (II): 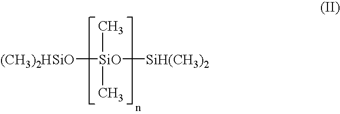
in which n is an integer greater than 1 and preferably greater than 10, for example ranging from 5 to 30, preferably from 10 to 30 and better still from 15 to 25. According to one particular embodiment of the invention, n is equal to 20.

The silicone block copolymers used according to the invention are advantageously free of oxyalkylene groups, especially free of oxyethylene and/or oxypropylene groups.

The catalyst for the reaction between the polysiloxane and the organosilicon compound may be chosen from metals and especially from platinum, rhodium, tin, titanium, copper and lead. It is preferably platinum or rhodium.

The dispersion of silicone copolymer particles used in the composition according to the invention may be obtained especially, for example, by mixing (a) water, (b) at least one emulsifier, (c) the polysiloxane (i), (d) the organosilicon compound (ii) and (e) a catalyst. Preferably, one of the constituents (c), (d) or (e) is added at the end to the mixture, in order for the chain-extension reaction to begin only in the dispersion.

As emulsifiers that may be used in the preparation process described above to obtain the aqueous particle dispersion, mention may be made of nonionic or ionic (anionic, cationic or amphoteric) emulsifiers. They are preferably nonionic emulsifiers, which may be chosen from polyalkylene glycol ethers of fatty alcohols, containing from 8 to 30 carbon atoms and preferably from 10 to 22 carbon atoms; polyoxyalkylenated and especially polyoxyethylenated sorbitan alkyl esters, in which the alkyl radical contains from 8 to 30 carbon atoms and preferably from 10 to 22 carbon atoms; polyoxyalkylenated and especially polyoxyethylenated alkyl esters, in which the alkyl radical contains from 8 to 30 carbon atoms and preferably from 10 to 22 carbon atoms; polyethylene glycols; polypropylene glycols; diethylene glycols; and mixtures thereof. The amount of emulsifier(s) is generally from 1% to 30% by weight relative to the total weight of the reaction mixture.

The emulsifier used to obtain the aqueous particle dispersion is preferably chosen from polyethylene glycol ethers of fatty alcohols and mixtures thereof, and especially polyethylene glycol ethers of alcohols containing 12 or 13 carbon atoms and from 2 to 100 oxyethylene units and preferably from 3 to 50 oxyethylene units, and mixtures thereof. Examples that may be mentioned include C₁₂-C₁₃ Pareth-3 and C₁₂-C₁₃ Pareth-23, and mixtures thereof.

According to one particular embodiment of the invention, the dispersion of silicone copolymer particles is obtained from dimethylvinylsiloxypolydimethylsiloxane (or divinyl dimethicone) as compound (i), and from the compound of formula (II) preferably with n=20, as compound (ii), preferably in the presence of a catalyst of platinum type, and the particle dispersion is preferably obtained in the presence of C₁₂-C₁₃ Pareth-3 and C₁₂-C₁₃ Pareth-23 as emulsifiers.

A dispersion of silicone copolymer particles that may especially be used is the product sold under the name HMW 2220 by the company Dow Corning (CTFA name: divinyl dimethicone/dimethicone copolymer/C₁₂-C₃ Pareth-3/C₁₂-C₁₃ Pareth 23), which is an aqueous 60% dispersion of divinyl dimethicone/dimethicone copolymer containing C₁₂-C₁₃ Pareth-3 and C₁₂-C₁₃ Pareth-23, the dispersion comprising about 60% by weight of copolymer; 2.8% by weight of C₁₂-C₁₃ Pareth-23; 2% by weight of C₁₂-C₁₃ Pareth-3; 0.31% by weight of preserving agents, the remainder to 100% being water.

The amount of particles of substantially linear block silicone copolymer may range, for example, from 0.01% to 15% by weight of active copolymer material, preferably from 0.05% to 10% by weight and better still from 0.05% to 5% by weight relative to the total weight of the composition, which represents, for example, an amount of HMW 2220 of from 0.01% to 25% by weight, preferably from 0.08% to 17% by weight and better still from 0.08% to 8.3% by weight relative to the total weight of the composition, since this starting material contains 60% by weight of copolymer.

Elastomeric Organopolysiloxane

The composition of the invention contains at least one elastomeric organopolysiloxane, which is preferably at least partially crosslinked. The term “elastomeric” means a soft, deformable solid material with viscoelastic properties especially having the consistency of a sponge or a soft sphere. Its modulus of elasticity is such that this material withstands deformation and has limited stretchability and contractability. This material is capable of regaining its original shape after stretching. This elastomer is formed from polymer chains of high molecular weight, the mobility of which is limited by a uniform network of crosslinking points.

The elastomeric organopolysiloxanes used in the composition according to the invention are preferably partially or totally crosslinked. They are in the form of particles. In particular the elastomeric organopolysiloxane particles range from 0.1 to 500 μm, preferably from 3 to 200 μm and better still from 3 to 50 μm in size. These particles may have any shape, for example they may be spherical, flat or amorphous.

When they are included in an oily phase, these elastomeric organopolysiloxanes become transformed, depending on the content of oily phase used, into a product of spongy appearance when they are used in the presence of small amounts of oily phase, or into a homogeneous gel in the presence of larger amounts of oily phase. The gelation of the oily phase with these elastomers may be total or partial.

Thus, the elastomers of the invention may be conveyed in the form of an anhydrous gel consisting of an elastomeric organopolysiloxane and of an oily phase. The oily phase used in the manufacture of the anhydrous gel of elastomeric organopolysiloxane contains one or more oils that are liquid at room temperature (25° C.) chosen from hydrocarbon-based oils and/or silicone oils. Advantageously, the oily phase is a silicone liquid phase, containing one or more oils chosen from polydimethylsiloxanes containing a linear or cyclic chain, which are liquid at room temperature, optionally comprising an alkyl or aryl chain that is pendent or at the end of the chain, the alkyl chain containing from 1 to 6 carbon atoms.

The elastomeric organopolysiloxanes used according to the invention may be chosen from the crosslinked polymers described in patent application EP-A-0 295 886 and from those described in patent U.S. Pat. No. 5,266,321.

They are preferably elastomeric organopolysiloxanes obtained by addition and crosslinking reaction, in the presence of a catalyst, preferably a catalyst of the platinum type, of at least:
(a) one organopolysiloxane (iii) containing two vinyl groups in □-ω position on the silicone chain per molecule; and
(b) one organopolysiloxane (iv) containing at least two hydrogen atoms linked to a silicon atom per molecule.

The first organopolysiloxane (iii) is chosen from polydimethylsiloxanes; it is preferably an, α-ω-dimethylvinyl-polydimethylsiloxane.

The organopolysiloxane is in the form of a gel obtained according to the following steps:
(a) mixing of the first and second organopolysiloxanes (iii) and (iv);
(b) adding an oily phase to the mixture from step (a);
(c) polymerizing the first and second organopolysiloxanes (iii) and (iv) in the oily phase in the presence of a catalyst, preferably a platinum catalyst.

The elastomeric organopolysiloxanes used in the composition of the invention may be, for example, those sold under the names: KSG 6 by the company Shin-Etsu; Trefil E-505C or Trefil E-506C by the company Dow Corning; Gransil (SR-CYC, SR DMF10, SR-DC556) by the company Grant Industries, or those sold in the form of ready-made gels: KSG 15, KSG 16, KSG 17, KSG 18, KSG 26A or KSG 26B from the company Shin-Etsu; Gransil SR 5CYC gel, Gransil SR DMF 10 gel and Gransil SR DC556 gel from the company Grant Industries; 1229-02-167 and 1229-02-168 from the company General Electric. A mixture of silicone elastomers, and especially a mixture of these commercial products, may also be used.

The elastomeric organopolysiloxane used in the composition of the invention is preferably in the form of an anhydrous gel, and especially of an anhydrous gel formed from non-spherical particles of elastomeric organopolysiloxane, such as the KSG products. The elastomeric organopolysiloxane is preferably introduced into the oily phase of the emulsion according to the invention.

The elastomeric organopolysiloxane(s) used according to the invention are preferably present in an amount of active material ranging from 2% to 20% by weight and better still from 2% to 10% by weight relative to the total weight of the composition.

Filler

The term “filler” means any finely divided solid. It may be chosen from organic fillers and mineral fillers, and mixtures thereof.

Depending on the case, the filler may be introduced into the composition after mixing together the other constituents, and, for example, in the case of an emulsion, after preparing the emulsion, or alternatively, if an oily phase is present, in the oily phase of the composition. It may also be introduced during the preparation of the emulsion, into the aqueous phase or into the oily phase.

Organic Filler

As organic fillers (also known as organic powders) that may be used in the composition of the invention, examples that may be mentioned include polyamide particles and especially the powder Nylon 12, for instance the products sold under the name Orgasol by the company Atochem; polyethylene powders and beads such as those sold under the name Acumist (Acumist B-6 and Acumist B-12) by the company Allied and those sold under the name Microthene by the company Equistar; microspheres based on acrylic or methacrylic copolymers, such as those made of ethylene glycol dimethacrylate/lauryl methacrylate copolymer, sold by the company Dow Corning under the name Polytrap, or those made of methyl methacrylate/ethylene glycol dimethacrylate copolymer, sold under the name Microspheres M305 by the company Matsumoto; the polymethyl methacrylate microspheres sold under the name Microphere M100 by the company Matsumoto or under the name Covabead (for example Covabead LH 85 with a size of from 10 to 12 microns) by the company Wackherr; ethylene-acrylate copolymer powders, such as those sold under the name Flobeads by the company Sumitomo Seika Chemicals; expanded powders, such as hollow microspheres and especially the microspheres sold under the name Expancel by the company Kemanord Plast or under the name Micropearl F 80 ED by the company Matsumoto; powders of natural organic materials such as maize starch, wheat starch or rice starch, which may or may not be crosslinked, such as the starch powders crosslinked with octenyl succinate anhydride, sold under the name Dry-Flo by the company National Starch; silicone resin microbeads such as those sold under the name Tospearl (for example Tospearl 2000 B of about 6 microns in size) by the company Toshiba Silicone; and mixtures thereof.

Mineral Fillers

The mineral fillers used in the composition of the invention may be very varied. They may be chosen especially from talc; kaolin; boron nitride; metal oxides; micas; nacres; silica (or silicon dioxide) powders; bismuth oxychloride; zinc stearate; particles of an alkali metal or alkaline-earth metal salt, for instance calcium carbonate, barium sulphate or calcium sulphate particles; platinum particles; aluminas (especially active aluminas); aluminosilicates (especially clays); mixed silicates of alkali metals and/or alkaline-earth metals (smectites, Laponites, in particular Laponite DS, D, XLS or XLG sold by the company Laporte Industries Ltd., Saponites, in particular Veegum sold by the company Vanderbilt; zeolites; magnesia; composite materials based on mineral fillers; and mixtures thereof.

Examples of metal oxides that may be mentioned include titanium oxide (amorphous titanium dioxide or titanium dioxide crystallized in rutile and/or anatase form), zinc oxide, iron oxide, zirconium oxide and cerium oxide, or mixtures thereof. These metal oxides may be in the form of particles of micrometric or nanometric size with a mean particle size ranging, for example, from 5 to 100 nm. These metal oxides may be coated and may especially have a hydrophobic coating. The coated metal oxides that may be used in the composition of the invention may, for example, have undergone one or more treatments with one or more compounds chosen from alumina, silica, aluminium derivatives (for example stearate and laurate), silicon compounds (for example silicones, polydimethylsiloxanes, alkoxysilanes or siloxysilicates), sodium compounds, iron oxides, iron esters (for example stearate), fatty acids, fatty alcohols and derivatives thereof (such as stearic acid, hydroxystearic acid, stearyl alcohol, hydroxystearyl alcohol, lauric acid and derivatives thereof), lecithin, waxes (for example carnauba wax), (meth)acrylic polymers (for example polymethyl methacrylates) and fluoro compounds (for example perfluoroalkyl compounds and perfluoroalkyl ethers). The oxides may also be treated with a mixture of these compounds or they may comprise several successive coats.

Nacres may also be used. The term “nacres” should be understood as meaning iridescent particles that reflect light. Among the nacres that may be envisaged, mention may be made of natural mother-of-pearl, mica coated with titanium oxide, iron oxide, aluminium hydroxide, magnesium hydroxide, silica, natural pigment and bismuth oxychloride, and also coloured titanium mica, and mixtures thereof.

The silica that may be used as mineral filler may be chosen from silica microspheres, for instance those sold under the name Sunsphere H-51 by the company Asahi Glass, or under the name Silica Beads by the company Maprecos.

The hydrophilic or hydrophobic silicas sold by the company Degussa-Hüls under the names Aerosil 90, Aerosil 130, Aerosil 150, Aerosil 200, Aerosil 300, Aerosil 380, Aerosil OX 50, Silice FK 320 DS, Aerosil R202, Aerosil R805, Aerosil R812, Aerosil R972 and Aerosil R974 may also be used.

It is also possible to use silica as an aqueous dispersion, for example a dispersion of colloidal silica, such as the product sold under the name Bindzil 30/220® by the company Eka Chemicals, which is a colloidal dispersion of amorphous silica (size: 14 nanometres) in water (30/70), or the product sold under the name Cosmo S-40 by the company Catalysts Chemicals (size: 18 nanometres).

The silica may also consist of a particle totally or partially coated with silica, especially a mineral particle totally or partially coated with silica, such as the silica beads containing titanium oxide sold under the name Torayceram S-IT® by the company Toray; the silica-alumina microspheres containing titanium oxide (size: 105 μm) sold under the name Z-Light-Sphere W 1012® by the company Zeelan; the amorphous precipitated synthetic silica/titanium oxide particles (size: 106-500 μm), sold under the name Neosil PC20S® by the company Crosfield; the Nylon-6-silica-titanium oxide fibres (length of 2 mm and thickness of 2 denier) sold under the name Fiberlon Y2® by the company Wackherr; the silica coated with titanium dioxide and covered with porous silica (85/5/10) (size: 0.6 μm), sold under the name ACS-0050510® by the company SACI-CFPA; the alumina- and silica-treated anatase nanotitanium oxide at 40% in water (size: 60 nm, monodisperse), sold under the name Mirasun TIW 60® by the company Rhodia Chemie CRA; the 15/5/3 silica/alumina/cerium IV-coated anatase nanotitanium oxide (60 nm) as an aqueous 32% dispersion, sold under the name Mirasun TIW 160® by the company Rhodia Chemie CRA; the alumina- and silica-treated anatase nanotitanium oxide (34/4.3/1.7) as an aqueous 40% dispersion, sold under the name Tioveil AQ-N® by the company Uniqema; the silica-coated nanotitanium oxide (66/33) (titanium dioxide particle size: 30 nm; silica thickness: 4 nm) sold under the name Maxlight TS-04® by the company Nichimen Europe PLC; and the silica-coated nanotitanium oxide (80/20) (titanium dioxide particle size: 30 nm; silica thickness: 2 nm) sold under the name Maxlight TS-042 by the company Nichimen Europe PLC. These particles may also have optical properties in the composition and on the skin.

The filler(s) (organic and/or mineral) used in the composition in accordance with the invention is (are) generally present in active material amounts ranging from 0.01% to 30% by weight, more preferably from 0.05% to 20% and even more preferably from 0.1% to 10% by weight relative to the total weight of the composition.

C₂-C₃ Primary Alcohol

The term “C₂-C₃ primary alcohol” means a linear or branched aliphatic lower monoalcohol containing 2 or 3 carbon atoms. According to one preferred embodiment of the invention, the primary alcohol used is ethanol.

The amount of lower alcohol may range, for example, from 2% to 20% by weight and preferably from 3% to 10% by weight relative to the total weight of the composition.

The compositions according to the invention may be in any formm, including any galenical form conventionally used for topical application, and especially in the form of gels, dispersions, emulsions of liquid or semi-liquid consistency of the milk type, obtained by dispersing a fatty phase (also known as an oily phase) in an aqueous phase (O/W) or conversely (W/O), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention, the composition is in the form of an emulsion and especially an O/W emulsion.

A subject of the present invention is also a composition for topical application in the form of an O/W emulsion containing an oily phase dispersed in an aqueous phase, the composition containing at least one filler, the oily phase containing at least one elastomeric solid organopolysiloxane, the aqueous phase containing at least one C₂-C₃ primary alcohol and an aqueous dispersion of at least one substantially linear block silicone copolymer.

In addition, the compositions used according to the invention may be more or less fluid and may have, for example, the appearance of a white or coloured cream or gel. They may optionally be applied to the skin in aerosol form. They may also be in solid form, for example in the form of powder, in cast form, as a dish, or in the form of a stick. They are preferably in the form of a fluid cream.

The composition of the invention comprises an aqueous phase, which may consist solely of the aqueous dispersion of block silicone copolymer particles and of alcohol, but which generally comprises other water-soluble or water-dispersible constituents. In the substantially anhydrous compositions, i.e. compositions comprising less than 5% by weight of water relative to the total weight of the composition, the aqueous phase consists solely of the aqueous dispersion of block silicone copolymer particles and of primary alcohol. Depending on the galenical form of the composition, the amount of aqueous phase may range from 0.1% to 99% by weight, preferably from 0.5% to 98% by weight, better still from 30% to 95% by weight and even better still from 40% to 95% by weight relative to the total weight of the composition. This amount depends on the galenical form of the desired composition. The amount of water may represent all or some of the aqueous phase, and is generally at least 30% by weight relative to the total weight of the composition, especially when the composition is in emulsion form.

The compositions of the invention may contain, in the aqueous phase or in the oily phase if it comprises an oily phase, in addition to the primary alcohol, one or more hydrophilic, lipophilic and/or amphiphilic organic solvents that are physiologically acceptable, i.e. well tolerated and giving a cosmetically acceptable feel.

The organic solvents may represent from 0.5% to 50% and preferably from 2% to 20% of the total weight of the composition. The organic solvents may be chosen from the group consisting of hydrophilic organic solvents, lipophilic organic solvents and amphiphilic solvents, or mixtures thereof.

Among the organic solvents other than the primary alcohol that may be mentioned, for example, are polyols such as propylene glycol, isoprene glycol, butylene glycol, propylene glycol, glycerol or sorbitol; monoalkyl or dialkyl isosorbide in which the alkyl groups contain from 1 to 5 carbon atoms, for instance dimethyl isosorbide; polyethylene glycols, especially those containing from 6 to 80 ethylene oxides, such as polyethylene glycol 32 OE; ethylene glycol ethers, for instance diethylene glycol monomethyl or monoethyl ether; propylene glycol ethers, for instance dipropylene glycol methyl ether; polyol esters and ethers, such as polypropylene glycol (PPG) esters and more especially fatty acid esters of polypropylene glycol (PPG), fatty alkyl ethers of PPG, for instance PPG-23 oleyl ether and PPG-36 oleate; fatty acid alkyl esters, such as diisopropyl adipate, dioctyl adipate or alkyl benzoates; and mixtures thereof.

When the composition is in the form of an emulsion, the proportion of oily phase of the emulsion may range, for example, from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in a suitable manner depending on the emulsion to be obtained (W/O or O/W).

Examples of emulsifiers that may be mentioned for the W/O emulsions include dimethicone copolyols, such as the mixture of cyclomethicone and of dimethicone copolyol, sold under the name DC 5225 C by the company Dow Corning or the mixture sold under the name Abil EM 97 by the company Goldschmidt, and alkyldimethicone copolyols, such as the laurylmethicone copolyol sold under the name Dow Corning 5200 Formulation Aid by the company Dow Corning, the cetyldimethicone copolyol sold under the name Abil EM 90 by the company Goldschmidt, or the mixture of cetyldimethicone copolyol, polyglyceryl-4 isostearate and hexyl laurate, sold under the name Abil WE 09 by the company Goldschmidt. One or more co-emulsifiers may also be added thereto, which may be advantageously chosen from the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Examples of emulsifiers that may be mentioned for the O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; fatty alkyl ethers; the sugar ethers of C₈-C₂₄ fatty alcohols, and mixtures thereof.

Glyceryl esters of fatty acids that may especially be mentioned include glyceryl stearate (glyceryl mono-, di- and/or tristearate) (CTFA name: glyceryl stearate) or glyceryl ricinoleate, and mixtures thereof.

Polyethylene glycol esters of fatty acids that may especially be mentioned include polyethylene glycol stearate (polyethylene glycol mono-, di- and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

Mixtures of these surfactants may also be used, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (CTFA name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: Methyl glucose dioleate); the mixed ester of methylglucoside and of the oleic acid/hydroxystearic acid mixture (CTFA name: Methyl glucose dioleate/hydroxysterate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and of isostearic acid (CTFA name: Methyl glucose sesquiisostearate); the mixture of the monoester and diester of methylglucoside and of stearic acid (CTFA name: Methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate.

Examples of fatty alkyl ethers that may be mentioned include polyethylene glycol ethers of fatty alcohols containing from 8 to 30 carbon atoms and especially from 10 to 22 carbon atoms, such as polyethylene glycol ethers of cetyl alcohol, of stearyl alcohol or of cetearyl alcohol (mixture of cetyl alcohol and stearyl alcohol). Examples that may be mentioned include ethers comprising from 1 to 200 and preferably from 2 to 100 oxyethylene groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol 1 0 by the company Kao Chemicals, the product sold under the name Plantaren 2000 by the company Cognis, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Cognis; cocoglucoside, for instance the product sold under the name Plantacare 818/UP by the company Cognis; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Cognis; arachidyl glucoside, for example in the form of the mixture of arachidyl alcohol and behenyl alcohol and arachidyl glucoside, sold under the name Montanov 202 by the company SEPPIC; cocoylethylglucoside, for example in the form of the mixture (35/65) with cetyl alcohol and stearyl alcohol, sold under the name Montanov 82 by the company Seppic; and mixtures thereof.

When the composition is in the form of an emulsion, the nature of the oily phase of the emulsion is not critical. The oily phase may thus consist of any fatty substance and especially any oil conventionally used in cosmetics or dermatology. The oily phase generally comprises at least one oil, especially a cosmetic oil.

As oils that may be used in the composition of the invention, mention may be made for example of:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, coriander oil, arara oil, sunflower oil, castor oil, avocado oil, caprylic/capric acid triglycerides, for instance those sold by the company Stearineries Dubois or those sold under the names Miglyol 810, 812 and 818 by the company Dynamit Nobel, jojoba oil or shea butter oil;
synthetic esters and synthetic ethers, especially of fatty acids, for instance oils of formulae R^{a}COOR^{b} and R^{a}OR^{b} in which R^{a} represents the fatty acid residue containing from 8 to 29 carbon atoms and R^{b} represents a branched or unbranched hydrocarbon-based chain containing from 3 to 30 carbon atoms, such as, for example, purcellin oil, isononyl isononanoate, isopropyl myristate, 2-ethylhexyl palmitate; 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diiso-nonanoate; and pentaerythritol esters, for instance pentaerythrityl tetraisostearate;
substantially linear or branched hydrocarbons of mineral or synthetic origin, such as volatile or non-volatile liquid paraffins, and derivatives thereof, petroleum jelly, polydecenes, isohexadecane, isododecane and hydrogenated polyisobutene such as Parleam® oil;
fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;
alkoxylated fatty alcohols and especially ethoxylated fatty alcohols, such as oleth-12, ceteareth-12 and ceteareth-20;
partially hydrocarbon-based and/or partially silicone-based fluoro oils, for instance those described in document JP-A-2 295 912. Examples of fluoro oils that may also be mentioned include perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;
silicone oils, for instance volatile or non-volatile polymethylsiloxanes (PDMSs) containing a substantially linear or cyclic silicone chain, that are liquid or pasty at room temperature, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, that are pendant or at the end of a silicone chain, these groups containing from 2 to 24 carbon atoms; phenylsilicones, for instance phenyl-trimethicones, phenyldimethicones, phenyltrimethylsiloxydiphenylsiloxanes, diphenyldimethicones, diphenylmethyldiphenyltrisiloxanes, 2-phenylethyl-trimethysiloxysilicates and polymethylphenylsiloxanes;
mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, camauba wax or candelilla wax, paraffin waxes, lignite wax or microcrystalline waxes, ceresin or ozokerite, synthetic waxes such as polyethylene waxes, Fischer-Tropsch waxes; liquid petroleum paste; gums such as silicone gums (dimethiconol); silicone resins such as trifluoromethyl-C1-4-alkyldimethicone and trifluoropropyldimethicone.

These fatty substances may be chosen in a varied manner by a person skilled in the art so as to prepare a composition having the desired properties, for example in terms of consistency or texture.

According to one particular embodiment of the invention, the composition of the invention contains at least one silicone oil, preferably a volatile silicone oil, which may be chosen, for example, from cyclic or substantially linear polydimethylsiloxanes, and mixtures thereof. The cyclic polydimethylsiloxanes or cyclomethicones contain from about 3 to 9 silicon atoms and preferably from 4 to 6 silicon atoms and may be, for example, cyclohexadimethylsiloxane and cyclopentadimethylsiloxane. The volatile, substantially linear polydimethylsiloxanes preferably contain from about 3 to 9 silicon atoms. The volatile substantially linear polydimethylsiloxanes generally have a viscosity at 25° C. of less than or equal to 5 cSt, whereas the cyclomethicones generally have a viscosity at 25° C. of less than or equal to 10 cSt.

In a known manner, all the compositions of the invention may contain one or more adjuvants that are common in cosmetics and dermatology, hydrophilic or lipophilic gelling agents and/or thickeners; hydrophilic or lipophilic active agents; sequestering agents; antioxidants; preserving agents; acidifying or basifying agents; fragrances; film-forming agents; dyestuffs; and mixtures thereof.

The amounts of these various adjuvants are those conventionally used in the fields under consideration. In particular, the amounts of active agents vary according to the desired aim and are those conventionally used in the fields under consideration, for example from 0.1% to 20% and preferably from 0.5% to 10% relative to the total weight of the composition.

As hydrophilic gelling agents other than the polymers described above, examples that may be mentioned include carboxyvinyl polymers such as the Carbopol products (carbomers) and the Pemulen products (acrylate/C₁₀-C₃₀-alkylacrylate copolymer); polyacrylamides, for instance the crosslinked copolymers sold under the name Sepigel 305 (CTFA name: polyacrylamide/C13-14 isoparaffin/Laureth-7) or Simulgel 600 (CTFA name: acrylamide/sodium acryloyldimethyltaurate copolymer/isohexadecane/polysorbate 80) by the company SEPPIC; cellulose derivatives such as hydroxyethylcellulose; polysaccharides and especially gums such as xanthan gum; and mixtures thereof.

Lipophilic gelling agents that may be mentioned include modified clays such as hectorite and its derivatives, for instance the products sold under the name Bentone.

Active Agents

As active agents that may be used in the composition of the invention, examples that may be mentioned include moisturizers such as protein hydrolysates, sodium hyaluronate, polyols, for instance glycerol, glycols, for instance polyethylene glycols and sugar derivatives; natural extracts; emollients; free-radical scavengers; anti-inflammatory agents; procyannidol oligomers; vitamins, for instance vitamin A (retinol), vitamin E (tocopherol), vitamin C (ascorbic acid), vitamin B5 (panthenol), vitamin B3 (niacinamide), derivatives of these vitamins, especially esters thereof, and in particular vitamin C derivatives such as saccharide esters of ascorbic acid, for instance 2-ascorbylglucoside or 2-O-α-D-glucopyranosyl L-ascorbate, and metal salts of phosphorylated ascorbic acid, for instance magnesium ascorbyl phosphate; urea; caffeine; depigmenting agents such as kojic acid, hydroquinone and caffeic acid, salicylic acid and its derivatives; α-hydroxy acids such as lactic acid, glycolic acid and gluconic acid, and derivatives thereof, especially esters thereof and also salts thereof such as the gluconates, especially the magnesium, manganese, calcium and zinc gluconates; retinoids such as carotenoids, for instance α-carotene, β-carotene, zeaxanthin, cryptoxanthin, lutein and lycopene; sunscreens; hydrocortisone; melatonin; algal extracts, fungal extracts, plant extracts (for example extracts of meadow-sweet), yeast extracts and bacterial extracts; enzymes; steroids (for example dehydroepiandrosterone (or DHEA), and also its biological and chemical precursors and derivatives, and/or natural extracts containing them and especially extracts of Dioscorea plants, such as wild yam; antibacterial active agents, for instance 2,4,4′-trichloro-2′-hydroxydiphenyl ether (or triclosan), 3,4,4′-trichlorocarbanilide (or triclocarban) and the acids indicated above and especially salicylic acid and its derivatives; matting agents, for instance fibres; tensioning agents; and mixtures of these active agents.

As active agent, the composition may especially comprise a tensioning agent. The term “tensioning agent” means a compound capable of exerting tension on the skin, which has the effect of temporarily fading out the irregularities in the surface of the skin, such as wrinkles and fine lines. Among the tensioning agents that may be used in the composition according to the present invention, mention may be made especially of:
(1) synthetic polymers, such as polyurethane latices or acrylic-silicone latices, in particular those described in patent application EP-1 038 519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold especially by the company 3M under the trade names VS 80 (INCI name: Polysilicone 8), VS 5 70 or LO21,
(2) polymers of natural origin, especially (a) polyholosides, for example (i) in the form of starch derived especially from rice, from maize, from potato, from cassava, from pea, from Triticum aestivum wheat, from oat, etc. or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins and cellulose derivatives, and mixtures thereof,
(3) plant proteins and plant protein hydrolyzates, in particular from maize, from rye, from Triticum aestivum wheat, from buckwheat, from sesame, from spelt, from pea, from bean, from lentil, from soybean and from lupin,
(4) microparticles of wax chosen, for example, from camauba wax, candelilla wax or esparto grass wax,
(5) colloidal particles of mineral filler with a numerical mean diameter of between 0.1 and 100 nm, preferably between 3 and 30 nm, chosen, for example, from: silica, cerium oxide, zirconium oxide, alumina, calcium carbonate, barium sulphate, calcium sulphate, zinc oxide and titanium dioxide.

The UV-screening agents or photoprotective agents that may be used in the composition in accordance with the invention may be chosen from organic photoprotective agents and/or at least the mineral photoprotective agents that are UVA-active and/or UVB-active (absorbers), which are water-soluble or liposoluble, or else insoluble in the cosmetic solvents commonly used.

The organic photoprotective agents are especially chosen from anthranilates; cinnamic derivatives; dibenzoylmethane derivatives; salicylic derivatives; camphor derivatives; triazine derivatives such as those described in documents U.S. Pat. No. 4,367,390, EP-A-863 145, EP-A-517 104, EP-A-570 838, EP-A-796 851, EP-A-775 698, EP-A-878 469, EP-A-933 376, EP-A-507 691, EP-A-507 692, EP-A-790 243 and EP-A-944 624; benzophenone derivatives; β,β-diphenylacrylate derivatives; benzotriazole derivatives; benzalmalonate derivatives; benzimidazole derivatives; imidazolines; bis-benzazolyl derivatives as described in documents EP-A-669 323 and U.S. Pat. No. 2,463,264; p-aminobenzoic acid (PABA) derivatives; methylenebis(hydroxyphenylbenzotriazole) derivatives as described in documents U.S. Pat. No. 5,237,071, U.S. Pat. No. 5,166,355, GB-A-2 303 549, DE-A-197 26 184 and EP-A-893 119; screening polymers and screening silicones such as those described especially in document WO-A-93/04665; dimers derived from α-alkylstyrene such as those described in document DE-A-198 55 649.

As examples of UV-A-active and/or UV-B-active photoprotective agents, mention may be made more particularly of the following, denoted hereinbelow under their INCI name:
para-aminobenzoic acid derivatives, including the following: PABA, Ethyl PABA, Ethyl dihydroxypropyl PABA, Ethylhexyl dimethyl PABA sold in particular under the name “Escalol 507” by ISP, Glyceryl PABA, PEG-25 PABA sold under the name “Uvinul P25” by BASF;
salicylic derivatives, including the following: Homosalate sold in particular under the name “Neo Heliopan OS” by Haarmann and Reimer, Dipropylene glycol salicylate sold in particular under the name “Dipsal” by Scher, TEA salicylate sold in particular under the name “Neo Heliopan TS” by Haarmann and Reimer;
dibenzoylmethane derivatives, including the following: Butyl methoxydibenzoylmethane sold in particular under the trade name “Parsol 1789” by Hoffmann LaRoche, Isopropyldibenzoylmethane;
cinnamic derivatives, including the following: Ethylhexyl methoxycinnamate sold in particular under the trade name “Parsol MCX” by Hoffmann LaRoche, Isopropyl methoxycinnamate, Isoamyl methoxycinnamate sold in particular under the trade name “Neo Heliopan E 1000” by Haarmann and Reimer, Cinoxate, DEA methoxycinnamate, Diisopropyl methylcinnamate, Glyceryl ethylhexanoate dimethoxycinnamate;
β,β-diphenyl acrylate derivatives, including the following: Octocrylene sold in particular under the trade name “Uvinul N539” by BASF, Etocrylene sold in particular under the trade name “Uvinul N35” by BASF;
benzophenone derivatives, including the following: Benzophenone-1 sold in particular under the trade name “Uvinul 400” by BASF, Benzophenone-2 sold in particular under the trade name “Uvinul D50” by BASF, Benzophenone-3 or Oxybenzone sold in particular under the trade name “Uvinul M40” by BASF, Benzophenone-4 sold in particular under the trade name “Uvinul MS40” by BASF, Benzophenone-5, Benzophenone-6 sold in particular under the trade name “Helisorb 11” by Norquay, Benzophenone-8 sold in particular under the trade name “Spectra-Sorb UV-24” by American Cyanamid, Benzophenone-9 sold in particular under the trade name “Uvinul DS-49” by BASF, Benzophenone-12, n-hexyl 2-(4-diethylamino-2-hydroxybenzoyl)benzoate;
benzylidenecamphor derivatives, including the following: 3-Benzylidenecamphor, 4-Methylbenzylidenecamphor sold in particular under the name “Eusolex 6300” by Merck, Benzylidenecamphorsulfonic acid, Camphor benzalkonium methosulfate, Terephthalylidenedicamphorsulfonic acid (“Mexoryl SX” by Chimex), Polyacrylamidomethylbenzylidenecamphor;
benzimidazole derivatives, including the following: Phenylbenzimidazolesulfonic acid sold in particular under the trade name “Eusolex 232” by Merck, Disodium phenyldibenzimidazoletetrasulfonate sold in particular under the trade name “Neo Heliopan AP” by Haarmann and Reimer;
triazine derivatives, including the following: Anisotriazine sold in particular under the trade name “Tinosorb S” by Ciba Specialty Chemicals, Ethylhexyltriazone sold in particular under the trade name “Uvinul T150” by BASF, Diethylhexylbutamidotriazone sold in particular under the trade name “Uvasorb HEB” by Sigma 3V, 2,4,6-Tris(diisobutyl 4′-aminobenzalmalonate) s-triazine;
benzotriazole derivatives, including the following: Drometrizole trisiloxane sold under the name “Silatrizole” by Rhodia Chimie, Methylenebis(benzotriazolyl)tetramethylbutylphenol sold in particular in solid form under the trade name “MIXXIM BB/100” by Fairmount Chemical, or in micronized form as an aqueous dispersion under the trade name “Tinosorb M” by Ciba Specialty Chemicals;
anthranilic derivatives, including Menthyl anthranilate sold under the trade name “Neo Heliopan MA” by Haarmann and Reimer;
imidazoline derivatives, including Ethylhexyldimethoxybenzylidenedioxoimidazoline propionate;
benzalmalonate derivatives, including Polyorganosiloxane with benzalmalonate functions, sold under the trade name “Parsol SLX” by Hoffmann LaRoche;
   and mixtures thereof.

The organic photoprotective agents that are more particularly preferred are chosen from the following compounds: Ethylhexyl salicylate, Ethylhexyl methoxycinnamate, Octocrylene, Phenylbenzimidazolesulfonic acid, Benzophenone-3, Benzophenone-4, Benzophenone-5,4-Methylbenzylidenecamphor, Terephthalylidenedicamphorsulfonic acid, Disodium phenyldibenzimidazoletetrasulfonate, 2,4,6-Tris(diisobutyl 4′-aminobenzalmalonate)-s-triazine, Anisotriazine, Ethylhexyltriazone, Diethylhexylbutamidotriazone, Methylenebis(benzotriazolyl)tetramethylbutyl-phenol, Drometrizole trisiloxane, and mixtures thereof.

The mineral photoprotective agents may be chosen from pigments or even nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminium stearate. Such coated or uncoated metal oxide nanopigments are described in particular in patent applications EP-A-518 772 and EP-A-518 773. Examples that may be mentioned include the hydrophilic nanotitanium sold under the name Mirasun TIW60 by the company Rhodia, and the lipophilic nanotitanium sold under the name MT100T by the company Tayca or UV Titan M 160 by the company Kemira.

When they are present in the compositions according to the invention, the UV-screening agents are generally in proportions ranging from 0.1% to 20% by weight relative to the total weight of the composition, and preferably ranging from 0.2% to 15% by weight relative to the total weight of the composition.

The amount of active agents depends on the desired aim. The active agent(s) may, for example, be present in general in a concentration ranging from 0.001% to 20%, preferably from 0.01% to 10% by weight and better still from 0.05% to 5% relative to the total weight of the composition.

According to one preferred embodiment of the invention, the composition comprises at least one active agent chosen from tensioning agents such as grafted silicone polymers and especially Polysilicone 8; α-hydroxy acid salts such as α-hydroxy acid gluconates; carotenoids and especially lycopene; vitamin C derivatives and especially 2-ascorbyl glucoside and magnesium ascorbyl phosphate; sodium hyaluronate; plant extracts and especially extract of meadow-sweet; sweet; and mixtures thereof.

Needless to say, a person skilled in the art will take care to select the optional compound(s) to be added to the composition according to the invention and the amounts thereof, such that the advantageous properties intrinsically associated with the composition in accordance with the invention are not, or are not substantially, adversely affected by the envisaged addition.

The composition preferably has a pH that is gentle to the skin, which generally ranges from 3 to 8 and preferably from 4.5 to 7.

The composition according to the invention may especially constitute a cosmetic composition, and in particular a care product, a protective product and/or a makeup product for the skin, especially to give the skin a matt appearance, and more particularly to fade out skin relief defects such as microreliefs, wrinkles and pores while at the same time making the skin look natural.

Thus, another subject of the invention is the cosmetic use of a cosmetic composition as defined above to give the skin a matt appearance and/or to fade out skin relief defects such as microreliefs, wrinkles and pores while at the same time making the skin look natural.

The subject of the invention is also a cosmetic process for giving the skin a matt appearance and/or for fading out skin relief defects, which comprises applying to the skin, particularly skin in need thereof, a composition as defined above, preferably one comprising effective amounts of the noted ingredients to provide the desired effects.

The examples that follow serve to illustrate the invention without, however, being limiting in nature. The amounts therein are given as percentages by weight.

### EXAMPLE 1

### O/W Emulsion

| Phase A (aqueous phase) | |
|---|---|
| Glycerol | 0.5% |
| Manganese gluconate | 0.05% |
| Ascorbyl glucoside | 0.5% |
| Sodium hyaluronate | 0.1% |
| Ammonium polyacryldimethyl tauramide (Hostacerin AMPS) | |
| Acrylamide/sodium acryloyldimethyltaurate | 0.4% |
| copolymer/isohexadecane/polysorbate 80 (Simulgel 600) | |
| Magnesium aluminium silicate (Veegum) | |
| Preserving agent | qs % |
| Water | qsp 100% |
| Phase B (oily phase) | |
| Elastomeric organopolysiloxane (KSG 16 containing 24% | 15% |
| active material) | |
| (i.e. 3.6% active material) | |
| Cyclohexadimethylsiloxane | 9% |
| Tocopheryl acetate | 0.1% |
| Polymethylsilsesquioxane (Tospearl 2000 B) | 0.5% |
| Phase C | |
| Denatured ethanol | 10% |
| Phase D | |
| Fragrance | qs |
| Phase E | |
| Polymethyl methacrylate (Covabead LH 85) | 0.25% |
| Mica/titanium oxide (56/44) | 0.5% |
| Phase F | |
| HMW2220 (Dow Corning) | 2% |

Procedure: Phase A is prepared by heating, to about 80° C., the mixture of water, glycerol, preserving agent, sodium hyaluronate, ascorbyl glucoside and manganese gluconate, and dispersing therein the other starting materials of phase A (Hostacerin AMPS, Simulgel 600 and Veegum). Next, phase B preheated to 60° C. is added thereto with stirring. After cooling to room temperature, phases D, E and F are successively added with stirring.

A light, nacreous and very gentle fluid with a good matting effect is obtained.

Test:

A composition similar to that of Example 1 but not containing phase F (comparative example) was prepared and the two compositions were tested by ten individuals. The individuals applied the compositions to half the face (each composition on one half of the face) and noted the sensations perceived during the application of the product (softness, emollience, film-forming nature, dry effect). They were asked the same questions after drying of the product (about 2 minutes after application).

This test showed that the 10 individuals all noted that the composition of Example 1 according to the invention had a markedly improved emollient and film-forming effect relative to the composition of the comparative example, and that the soft effect of the composition containing KSG and fillers was well maintained.

### EXAMPLE 2

### O/W Emulsion

| Phase A (aqueous phase) | |
|---|---|
| Glycerol | 1% |
| Extract of meadow-sweet (Spiraea ulmaria) | 0.1% |
| Ammonium polyacryldimethyl tauramide (Hostacerin AMPS) | 0.7% |
| Acrylamide/sodium acryloyldimethyltaurate | 0.2% |
| copolymer/isohexadecane/polysorbate 80 (Simulgel 600) | |
| Magnesium aluminium silicate (Veegum) | 0.3% |
| Preserving agent | qs % |
| Water | qs 100% |
| Phase B (oily phase) | |
| Elastomeric organopolysiloxane (KSG 16 containing 24% | 15% |
| active material) | |
| (i.e. 3.6% active material) | |
| Cyclohexadimethylsiloxane | 9% |
| Polysilicone 8 | 1% |
| Dimethiconol (silicone gum) | 0.3% |
| Phase C | |
| Denatured ethanol | 5% |
| Phase D | |
| Fragrance | qs |
| Phase E | |
| Polymethyl methacrylate (Covabead LH 85) | 0.25% |
| Mica coated with aluminium hydroxide (65/35) | 0.25% |
| Phase F | |
| HMW2220 (Dow Corning) | 2% |

The procedure is similar to that of Example 1. A fluid that is soft on application and emollient is obtained.

The above written description of the invention provides a manner and process of making and using it such that any person skilled in this art is enabled to make and use the same, this enablement being provided in particular for the subject matter of the appended claims, which make up a part of the original description and including a composition for topical application, comprising at least one elastomeric solid organopolysiloxane, at least one filler, at least one C₂-C₃ primary alcohol and an aqueous dispersion of at least one substantially linear block silicone copolymer.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

As used herein, where a polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted and reactive “monomers,” but instead is made up of repeating units derived from reacted monomers.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition for topical application comprising at least one elastomeric solid organopolysiloxane, at least one filler, at least one C₂-C₃ primary alcohol and an aqueous dispersion of at least one substantially linear block silicone copolymer.

2. The composition according to claim 1, wherein the amount of block silicone copolymer ranges from 0.01% to 15% by weight relative to the total weight of the composition.

3. The composition according to claim 1, wherein the block silicone copolymer is in the form of particles having a number-average size of less than or equal to 2 microns.

4. The composition according to claim 1, wherein the block silicone copolymer is obtained by chain-extension reaction, in the presence of a catalyst, starting with at least:
(a) one polysiloxane (i) having at least one reactive group; and
(b) one organosilicon compound (ii) that reacts with the polysiloxane (i) by chain-extension reaction.

5. The composition according to claim 4, wherein the organopolysiloxane (i) is selected from the group consisting of the compounds of formula (I) and mixtures thereof: 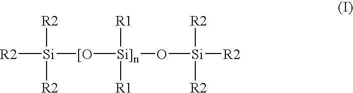
in which R₁ and R₂, independently of each other, represent a hydrocarbon-based group containing from 1 to 20 carbon atoms or an aryl group or a reactive group, n is an integer greater than 1, with the proviso that there are on average between one and two reactive groups per polymer.

6. The composition according to claim 5, wherein the reactive group is selected from the group consisting of hydrogen; aliphatically unsaturated groups; a hydroxyl group; alkoxy groups; alkoxyalkoxy groups; an acetoxy group; amino groups; and mixtures thereof.

7. The composition according to claim 5, wherein R₁ represents a methyl group.

8. The composition according to claim 5, wherein R₂ at the end of a chain represents a vinyl group.

9. The composition according to claim 4, wherein the organosilicon compound (ii) is selected from the group consisting of the polysiloxanes of formula (I), compounds acting as chain extenders, and mixtures thereof.

10. The composition according to claim 9, wherein compound (ii) is a liquid organohydrogenopolysiloxane of formula (II): 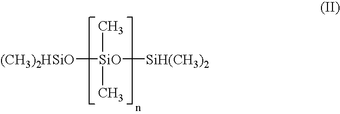
in which n is an integer greater than 10.

11. The composition according to claim 10, wherein, in formula (II), n is equal to 20.

12. The composition according to claim 4, wherein the aqueous dispersion of silicone copolymer particles is obtained by mixing water, at least one emulsifier, the polysiloxane (i), the organosilicon compound (ii) and a catalyst.

13. The composition according to claim 12, wherein the dispersion is an aqueous dispersion of divinyl dimethicone/dimethicone copolymer, C₁₂-C₁₃ Pareth-3 and C₁₂-C₁₃ Pareth-23.

14. The composition according to claim 1, wherein the elastomeric organopolysiloxane is obtained by addition reaction and crosslinking reaction, in the presence of a catalyst, of at least:
one first organopolysiloxane (iii) containing two vinylic groups in the α-ω position of the silicone chain per molecule; and
one second organopolysiloxane (iv) containing at least one hydrogen atom linked to a silicon atom per molecule.

15. The composition according to claim 14, wherein the first organopolysiloxane (iii) is an α-ω-dimethylvinyl polydimethylsiloxane.

16. The composition according to claim 14, wherein the organopolysiloxane is in the form of a gel obtained according to the following steps:
(a) mixing of the first and second organopolysiloxanes (iii) and (iv);
(b) addition of an oily phase to the mixture from step (a);
(c) polymerization of the first and second organopolysiloxanes (iii) and (iv) in oily phase in the presence of a platinum catalyst.

17. The composition according to claim 1, wherein the elastomeric organopolysiloxane is in the form of an anhydrous gel.

18. The composition according to claim 1, wherein the amount of elastomeric organopolysiloxane ranges from 2% to 20% by weight relative to the total weight of the composition.

19. The composition according to claim 1, wherein the filler is selected from the group consisting of organic fillers, mineral fillers, and mixtures thereof.

20. The composition according to claim 1, wherein the filler is an organic filler selected from the group consisting of polyamide particles; polyethylene powders and beads; microspheres based on acrylic or methacrylic copolymers; polymethyl methacrylate microspheres; ethylene-acrylate copolymer powders; expanded powders; powders of natural organic materials; silicone resin microbeads; and mixtures thereof.

21. The composition according to claim 1, wherein the filler is a mineral filler selected from the group consisting of talc; kaolin; boron nitride; metal oxides; micas; nacres; silica powders; bismuth oxychloride; zinc stearate; alkali metal or alkaline-earth metal salt particles; platinum particles; aluminas; aluminosilicates; mixed silicates of alkali metals and/or alkaline-earth metals; zeolites; magnesia; composite materials based on mineral fillers; and mixtures thereof.

22. The composition according to claim 1, wherein the amount of filler(s) ranges from 0.01% to 30% by weight relative to the total weight of the composition.

23. The composition according to claim 1, wherein the C₂-C₃ primary alcohol is ethanol.

24. The composition according to claim 1, wherein the C₂-C₃ primary alcohol is present in an amount ranging from 2% to 20% by weight relative to the total weight of the composition.

25. The composition according to claim 1, further comprising at least one active agent selected from the group consisting of tensioning agents, α-hydroxy acid salts, carotenoids, plant extracts, sodium hyaluronate, vitamin C derivatives, and mixtures thereof.

26. The composition according to claim 1, wherein the active agent is selected from the group consisting of grafted silicone polymers, α-hydroxy acid gluconates, lycopene, 2-ascorbylglucoside, magnesium ascorbylphosphate, sodium hyaluronate; extract of meadow-sweet, and mixtures thereof.

27. The composition according to claim 1, wherein it is in the form of an oil-in-water emulsion.

28. The composition according to claim 1, wherein isaid composition constitutes a care product, a protective product and/or a makeup product for the skin.

29. A method of providing the skin with a matt appearance and/or to fade out skin relief defects while at the same time making the skin look natural, comprising applying the composition of claim 1 to skin in need thereof.

30. A method of providing the skin with a matt appearance and/or to fade out skin relief defects while at the same time making the skin look natural, comprising applying the composition of claim 4 to skin in need thereof.
